(19) United States
(12) Patent Application Publication
(10) Pub. No.: US 2014/0179597 A1
(43) Pub. Date: Jun. 26, 2014

(54) Method for the treatment and prevention of Alzheimer's disease and central nervous system dysfunction

(71) Applicant: Steven Lehrer, New York, NY (US)

(72) Inventor: Steven Lehrer, New York, NY (US)

(21) Appl. No.: 14/071,458

(22) Filed: Nov. 4, 2013

Related U.S. Application Data
(60) Provisional application No. 61/727,266, filed on Nov. 16, 2012.

(51) Int. Cl. A61K 9/00; A61K 38/28

(52) U.S. Cl. CPC A61K 9/0043; A61K 38/28
USPC 514/5.9

(57) ABSTRACT

Intranasal insulin is beneficial for the treatment of Alzheimer's disease and other neurologic disorders. But commercial insulin preparations use metacresol, phenol, and other preservatives for chemical stabilization. Metacresol in particular has multiple toxic effects when administered long term, especially intranasal. These effects include rhinitis and nosebleeds. The present invention teaches the use of intranasal insulin not preserved with meta-cresol, phenol or other excipients for the treatment of Alzheimer's disease and other neurologic disorders.

This application claims priority to U.S. Provisional Application 61/727,266 filed Nov. 16, 2012. The entire disclosure of the application is incorporated by reference herein.

Alzheimer's disease (AD) is linked to CNS insulin resistance, decreased expression of insulin, insulin receptor genes, and lower cerebrospinal insulin levels. Impaired brain insulin signaling may account for some of the cognitive deficits associated with. Alzheimer's disease. Using, intranasal administration to deliver insulin directly to, the brain through, the olfactory neurons, a series of acute clinical trials involving healthy humans and AD patients have shown that increased CNS insulin action enhances learning and memory processes (Freiherr et al., 2013).

Other evidence comes from Pomytkin, who teaches the use intranasal pharmaceutical compositions for preventing and/or treating a disease selected from the group consisting of Alzheimer's disease, Parkinson's disease, cerebral ischemia and neurological damage due to stroke comprising a therapeutically effective amount of succinic acid or a pharmaceutically acceptable salt thereof, and a pharmaceutically and intranasal acceptable carrier, along with insulin (POMYTKIN, 2010). Sveinbörn Gizurarson teaches administering a unit dose of the therapeutic preparation by ejection from a nasal spray device through a nostril of a human, the device and the pharmaceutical preparation being adapted so that the spray angle is at the most 35 DEG. The method is useful for the treatment of diseases affecting the olfactory organ, the brain and the central nervous system. Sveinbörn Gizurarson's invention also relates to a nasal spray device comprising a pharmaceutical preparation. The pharmaceutical preparation preferably contains a viscosity enhancing agent and the pharmaceutical preparation preferably has a dynamic viscosity in the range of 5-300 cP (Sveinbörn Gizurarson, 2003). Touitou teach use of phospholipids, one or more C2-C4 alcohols and water in the preparation of a vesicular composition adapted for intranasal administration of an active agent such as insulin, wherein the concentrations of said phospholipids and said one or more alcohols in said composition are in the ranges of 0.2 to 10% and 12 to 30% by weight, respectively, with the water content of said composition being not less than 30% by weight (TOUITOU et al., 2012). Frey teaches a method for transporting neurologic therapeutic and/or diagnostic neurologic agents, such as insulin, to the brain by means of the olfactory neural pathway and a pharmaceutical composition useful in the treatment and diagnosis of brain disorders (FREY William, III, 1997). Frey also teaches that patients at risk for Alzheimer's or certain other diseases or disorders that are associated with risk for cerebral ischemia may benefit from intranasal insulin. Intranasal therapeutic agents are administered to the upper third of the nasal cavity to bypass the blood-brain barrier and access the central nervous system directly to avoid unwanted and potentially lethal side effects. Therapeutic agents include those substances that interact with iron and/or copper such as iron chelators, copper chelators, and antioxidants, for example, deferoxamine (DF( ) and deferasirox (FREY et al., 2010). Johansson (JOHANSSON, 2012) teaches that insulin aggregates in a nasal spray may be administered for treatment of Alzheimer's disease.

However, these and other methods suffer from a deficiency: Commercial insulin preparations contain meta-cresol, protamine, zinc, phenol, as well as other preservatives, to increase their chemical stability and lengthen shelf-life (Ghazavi and Johnston, 2011). Moreover, meta-cresol and phenol are added to injectable insulin preparations as anti-bacterial agents. Because the insulin is contained in multi-dose vials, there would be a high risk of bacterial contamination after a patient had inserted a needle into the vial multiple times. Meta-cresol and phenol are added to injectable insulin to lower this risk (Rathod et al., 1985).

Most exposures to cresols at very low levels are not harmful. But when cresols are breathed, ingested, or applied to the skin at higher levels, they can be harmful. Effects observed in humans include irritation and burning of skin, eyes, mouth, and throat; abdominal pain and vomiting; heart damage; anemia; liver and kidney damage; facial paralysis; coma; and death. Breathing high levels of cresols for a short time results in irritation of the nose and throat. Aside from these effects, little is known about the effects of breathing cresols, for example, at lower levels over longer times. Ingesting high levels results in kidney problems, mouth and throat bums, abdominal pain, vomiting, and effects on the blood and nervous system. Skin contact with high levels of cresols can bum the skin and damage the kidneys, liver, blood, brain, and lungs. Short-term and long-term studies with animals have shown similar effects from exposure to cresols. The effects of long-term ingestion or skin contact with low levels of cresols are unknown but may not be benign. The effect on olfaction of nasally instilled meta-cresol and/or phenol is unknown. Studies of intranasal insulin report rhinitis and nosebleeds, due to meta-cresol and phenol (Craft et al., 2012).

Patton teaches a preservative free insulin formulation that is specifically adapted for aerosolization and inhalation into the lungs for treatment of diabetes, but not nasal installation (Patton et al., 2011a; Patton et al., 2011b). Havelund teaches a meta-cresol and phenol-free aqueous preparation for inhalation into the lungs (Havelund, 2001) and also a lung inhalation preparation containing menthol to disguise the odor of meta-cresol and, phenol (Havelund, 2003).

The present invention uses preservative-free insulin, especially meta-cresol-free insulin and/or phenol-free insulin, for intranasal installation. The beneficial effect on Alzheimer's and other neurologic disorders is thus retained while avoiding the toxic and allergic effects of meta-cresol, phenol and other preservatives. The insulin could be administered nasally via pressurized aerosol, aqueous pump spray, powder, or other standard method (Thorsson et al., 1999). A second nasal formulation would comprise a standard insulin preparation stabilized with meta-cresol and/or phenol plus menthol, to disguise the odor of the meta-cresol and/or phenol. The daily dosage would be 20 International Units (IU) to 40 IU. Insulin-like growth factor and/or other growth factors could be added.

In a third formulation, a hand-held, metered, non-pressurized aerosol device (e.g. Pfeiffer pump, Boehringer, Ingelheim, FRG) that delivered insulin spray could be used. The nasal insulin formulation is prepared from crystals of lyophilized human recombinant insulin (Sigma Aldrich) to a final concentration of 250 U/ml, together with 1% (wt/vol) sodium glycocholate and 0.14 M phosphate buffer, pH 7.0. Methyl p-hydroxybenzoate (0.1% wt/vol) is used as a preservative. Stability studies have demonstrated that there is no statistically significant loss of insulin immunoreactivity with storage at 4° C. for up to 2 months. After 6 months, there is a 10-20% loss (Frauman et al., 1987).

### REFERENCE LIST

Craft S, Baker L D, Montine T J, Minoshima S, Watson G S, Claxton A, Arbuckle M, Callaghan M. Tsai E, Plymate S R, Green P S, Leverenz J, Cross D, Gerton B (2012) Intranasal insulin therapy for Alzheimer disease and amnestic mild cognitive impairment: a pilot clinical trial. Arch Neurol 69 (1): 29-38
Frauman A G, Cooper M E, Parsons B J, Jerums G, Louis W J (1987) Long-term use of intranasal insulin in insulin-dependent diabetic patients. Diabetes Care 10 (5): 573-578
Freiherr J, Hallschmid M, Frey W H, Brunner Y F, Chapman C D, Holscher C, Craft S, De Felice F G, Benedict C (2013) Intranasal insulin as a treatment for Alzheimer's disease: a review of basic research and clinical evidence. CNS Drugs 27 (7): 505-514
FREY William, H., III. NEUROLOGIC AGENTS FOR NASAL ADMINISTRATION TO THE BRAIN. 91901158[EP 0504263 B1]. Aug. 13, 1997. EP, EP-A-0 145 209; EP-A-0 351 808; FR-A-2 260 329; WO-A-86/04233; WO-A-89/01343. Dec. 4, 1990. Ref Type: Patent
FREY. I. I., PANTER, Samuel Scott, BRESIN HANSON, Leah Ranae U S, and ROEYTENBERG, Annina. METHODS AND PHARMACEUTICAL COMPOSITIONS FOR DIFFERENTIALLY ALTERING GENE EXPRESSION TO PROVIDE NEUROPROTECTION FOR THE ANIMAL CENTRAL NERVOUS SYSTEM AGAINST THE EFFECTS OF ISCHEMIA, NEURODEGENERATION, TRAUMA AND METAL POISONING. 12829844[U.S. 2010/0267834 A1]. Oct. 21, 2010. U.S. Jul. 2, 2010. Ref Type: Patent
Ghazavi M K, Johnston G A (2011) Insulin allergy. Clin. Dermatol 29 (3): 300-305
Havelund, S. Insulin preparations for pulmonary delivery containing menthol. 09418778[U.S. Pat. No. 6,635,617]. Oct. 21, 2003. U.S., EP 0692489 (Jan, 1996) ; U.S. Pat. No. 5,474,978 A (Dec, 1995) Bakaysa et al. 514/4; U.S. Pat. No. 5,506,203 A (Apr, 1996) Backstrom et al. 514/4; U.S. Pat. No. 5,743,250 A (Apr, 1998) Gonda et al. 128/200.14; U.S. Pat. No. 5,747,445 A (May, 1998) Backstrom et al. 514/4; U.S. Pat. No. 6,017,545 A (Jan, 2000) Modi 424/400; WO 9811867 (Mar, 1998); WO 96/40089 (Dec, 1996); WO 97/48413 (Dec, 1997); WO 98/42368 (Oct, 1998). Oct. 15, 1999. Ref Type: Patent
Havelund, S. Stable concentrated insulin preparations for pulmonary delivery. 09419668[U.S. Pat. No. 6,211,144]. Apr. 3, 2001. U.S., DE 29 52 119 A1 (Jul, 1981); EP 0692489A1 (Jan, 1996) ; U.S. Pat. No. 5,474,978 (Dec, 1995) Bakaysa et al. 514/4; U.S. Pat. No. 5,506,203 (Apr, 1996) Backstrom et al. 514/4; U.S. Pat. No. 5,743,250 (Apr, 1998) Gonda et al. 128/200.14; U.S. Pat. No. 5,747,445 (May, 1998) Backstrom et al. 514/4; U.S. Pat. No. 5,783,556 (Jul, 1998) Clark et al. 514/4; U.S. Pat. No. 5,830,999 (Nov, 1998) Dunn 530/303; U.S. Pat. No. 5,866,538 (Feb, 1999) Norup et al. 514/3; U.S. Pat. No. 5,952,297 (Sep, 1999) De Felippis et al. 514/3; U.S. Pat. No. 6,034,054 (Jul, 1998) DeFelippis et al. 514/4: WO 97/48413 (Dec, 1997) ; WO 98/42367 (Oct, 1998) ; WO 98/42368 (Oct, 1998). Oct. 15, 1999. Ref Type: Patent
JOHANSSON, Jan. Compound And Method For Treatment Of Alzheimer's Disease. 13145096[U.S. 2012/0122794 A1], May 17, 2012. U.S. Jan. 29, 2010. Ref Type: Patent
Patton, John S., PATTON, Ryan S., KUO, Mei chang, and IVRI, Yehuda. PRESERVATIVE FREE INSULIN FORMULATIONS AND SYSTEMS AND METHODS FOR AEROSOLIZING. 13004645[U.S. 2011/0168170 A1], Jul. 14, 2011a. U.S. Jan. 11, 2011a. Ref Type: Patent
Patton, John S., PATTON, Ryan S., K U O, Mei chang, and IVRI, Yehuda. PRESERVATIVE-FREE SINGLE DOSE INHALER SYSTEMS. 13004662[US 2011/0168172 A1]. Jul. 14, 2011b. US. Jan. 11, 2011b. Ref Type: Patent
POMYTKIN, Igor Anatolievich. INTRANASAL PHARMACEUTICAL COMPOSITIONS COMPRISING SUCCINIC ACID AND METHODS THEREOF. 12670690[U.S. 2010/0160440 A1]. Jun. 24, 2010. US. Aug. 2, 2007. Ref Type: Patent

Rathod M, Saravolatz. L, Pohlod D, Whitehouse F, Goldman J (1985) Evaluation of the sterility and stability of insulin from multidose vials used for prolonged periods. Infect Control 6 (12): 491-494
Sveinbörn Gizurarson, Kaplaskjolsvegur. Method for administration of active substances to the olfactory region. 09446931[U.S. Pat. No. 6,647,980], Nov. 18, 2003. U.S., EP 0452728 (Oct, 1991); EP 0734969 (Oct, 1996); FR 2130130 (Nov, 1972); U.S. Pat. No. 3,762,409 A (Oct, 1973) Lester 128/200.14; U.S. Pat. No. 4,054,622 A (Oct, 1977) Lester 128/200.14; U.S. Pat. No. 5,116,315 A (May, 1992) Capozzi et al.; U.S. Pat. No. 5,284,132 A (Feb, 1994) Geier 128/200.22; U.S. Pat. No. 5,630,793 A (May, 1997) Rowe 604/20; U.S. Pat. No. 5,683,361 A (Nov, 1997) Elk et al. 604/58; U.S. Pat. No. 5,921,233 A (Jul, 1999) Gold et al. 128/200.22; WO 93/02729 (Feb, 1993). Feb. 11. 2000. Ref Type: Patent
Thorsson L, Borga O, Edsbacker S (1999) Systemic availability of budesonide after nasal administration of three different formulations: pressurized aerosol, aqueous pump spray, and powder. Br J Clin Pharinacol 47 (6): 619-624
TOUITOU, Elka, GODIN, Biana, and DUCHI, Shaher. COMPOSITIONS FOR NASAL DELIVERY. 06796170.6[EP 1933809 Bi]. 2-8-2012. EP, U.S. Pat. No. 5,716,638; U.S.-A1-2002 048 551; U.S. Pat. No. 6,350,458; WO-A-01/06987; WO-A-03/048167. Oct. 15, 2006. Ref Type: Patent

What is claimed:

1. A method of achieving a therapeutically effective brain level of insulin comprising administering a dose of a pharmaceutical formulation of insulin or an analogue thereof into a single nostril or both nostrils.

2. Use according to claim 1 wherein a dose comprises at least about 10 international units (U) to about 100 U of insulin per 100 microliters.

3. Use according to claim 1, wherein the active substance of the pharmaceutical preparation is preservative-free insulin or insulin free of meta-cresol. phenol and/or other preservatives.

4. Use according to claim ,1 wherein the insulin in the pharmaceutical preparation is in the form of a dry powder or dissolved in water.

5. Use according to claim 1 wherein the pharmaceutical preparation is administered at least once per day.

6. Use according to claim 1 of a nozzle of a spray device to administer a unit dose of the pharmaceutical preparation by ejection through a nostril of a mammal to the olfactory region.

7. Use according to claim 1 for the treatment of Alzheimer's disease or other disease of the central nervous system.
